(19) United States
(12) Patent Application Publication
(10) Pub. No.: US 2010/0210956 A1
(43) Pub. Date: Aug. 19, 2010

(54) APPARATUS AND METHOD FOR SENSING RADIAL ARTERIAL PULSES FOR NONINVASIVE AND CONTINUOUS MEASUREMENT OF BLOOD PRESSURE AND ARTERIAL ELASTICITY

(75) Inventor: JAE JOONG IM, JEONJU (KR)

Correspondence Address: LRK Patent Law Firm, 1952 Gallows Rd, Suite 200, Vienna, VA, 22182, US

(73) Assignee: HANBYUL MEDITECH CO., LTD., JEONJU (KR)

(21) Appl. No.: 12/707,954

(22) Filed: Feb. 18, 2010

(30) Foreign Application Priority Data
May 23, 2008 (KR) 10-2008-0048006

(51) Int. Cl. A61B 5/022

(52) U.S. Cl. 600/490

(57) ABSTRACT

Provided is a radial arterial pulse sensing apparatus for noninvasive and continuous measurement of blood pressure and arterial elasticity. The apparatus includes two pressure sensor for detecting radial arterial pulses, two cuffs that are disposed under the respective pressure sensors and expandable by application of external pressure, a motor unit for providing proper pressure to expandable pouches under the respective pressure sensors in a state where a wrist band having the pressure sensors is put on a wrist, a pulse wave velocity calculating unit that calculates a pulse wave transfer velocity to attain a blood pressure value using an output from the pressure sensors, and an augmentation index calculating unit that estimates the blood pressure value by finding a time point of a reflective wave of the pulses using the outputs from the pressure sensors.

### BACKGROUND OF THE INVENTION

The present invention disclosed herein relates to a system for providing quantitative values for a brachial arterial blood pressure, a central arterial blood pressure, a blood vessel elasticity, a blood vessel aging, an estimated blood vessel age, an early diagnosis of a specific disease related to a cardiovascular system, and a disease possibility in the future by detecting an arterial pulse occurring in a radial artery of a human body, and more particularly, to a radial arterial pulse sensing apparatus and method, in which a pressure sensor and a cuff are applied to a radial artery of a wrist.

An artery blood pressure is determined by an amount of blood, an elasticity of a blood vessel, a contraction resistance, and the like. The artery blood pressure is one of vital signs representing disorder or inappropriateness of a cardiovascular system. The artery blood pressure is a major factor which affects vessel flows of all of organics and adjusts a cerebral blood flow or a coronary flow. The high blood pressure causes arteriosclerosis, a cardiovascular disease, a cerebrovascular disease, and a renal disease. The high blood pressure is caused by the increase of a peripheral blood vessel resistance and a low internal moisture flow. Particularly, the high blood pressure is mainly caused by exacerbation of the peripheral blood vessel resistance, i.e., contraction of a peripheral arteriole.

However, even when the blood vessel resistance increases, a high pressure value may not be appeared in a case where a pumping amount of a left ventricle. That is, there is no doubt that it is important to always monitor values of a brachial blood pressure and a central aortic blood pressure. However, a blood vessel resistance value and a blood vessel value are also important indicators to be always monitored. Further, the elasticity of the blood vessel is also an independent, important factor determining the blood pressure value.

Methods for measuring the blood vessel can be classified into an invasive method and a noninvasive method. The invasive method is generally used for treating critical patients in an intensive care unit or an operating room to continuously monitor the arterial blood pressure of the patients by attaining the arterial blood frequently for analyzing arterial blood gas. However, the invasive method is inconvenient to prepare and operate and may lead to respiratory complications due to infection or vein occlusion. Therefore, the application objects of the invasive method are mostly limited to the critical patients and careful management is required.

A method for measuring the blood pressure value by heating Korotkoff-sound using a cuff and an oscillometric method using an electronic automatic blood pressure gauge are generally used as daily measuring methods. However, these methods cannot continuously monitor the blood pressure and there may be a difference depending on a personal subject. Furthermore, when the blood pressure is reduced to a predetermined level, it is difficult to measure the blood pressure. Particularly, it is difficult to apply these methods to children or elderly persons. Furthermore, the accuracy of these methods is significantly reduced for the patients having a diastolic blood pressure less than 70 mmHg. It is troublesome to consider the circumference of a patient's hand when using the cuff. Furthermore, when the cuff is used, it is required to apply a pressure of about 200 mmHg, the blood vessel or tissues may be damaged. In order to solve these limitations, noninvasive, continuous blood pressure measurements without using the cuff have been attempted. However, the substantially reliable blood pressure monitoring is not yet realized.

Korean Pat. No. 10-0467056 discloses a non-invasive apparatus and method for automatically measuring blood pressure and Korean Pat. No. 10-0430144 discloses an electronic blood pressure measuring apparatus. However, the apparatuses and methods of these patents are not designed to continuously measure the blood pressure and to use the cuff causing the many limitations such as a blood pressure range limitation and unpleasant use. In order to accurately calculate the accurate blood pressure value, other vital signals such as electrocardiogram or oxygen saturation at an ear or finger are attained by attaching pads on the body. The user feels inconvenience by the pads.

U.S. Pat. No. 6,413,223 discloses a cuffless continuous blood pressure monitor and U.S. Pat. No. 6,669,648 discloses a continuous noninvasive sphygmomanometer. However, the measuring portion of these apparatuses is limited to the finger. In addition, additional devices for operating a light source are required to monitor the continuous blood pressure value. These devices are attached to the human body and thus the user feels inconvenience.

Thus, in order to conveniently measure the continuous blood pressure value without using the cuff but using a pulse of a radial artery of a wrist, a sensor should be properly arranged and selected considering an attaching surface of the human body. In addition, a proper correction should be done depending on conditions of the user. Further, the blood pressure value should be calculated by the pulse attained. In addition, the wearing and separation can be conveniently done.

When the pulse waveform of the radial artery is detected by an accurate, effective sensing method, an aortic waveform of the center of the heart is assumed from the attained radial arterial pulse waveform and an augmentation index of the aorta and the central aortic blood pressure value can be calculated using the assumed waveform. In order to estimate the elasticity of the artery, a difference between a value at a time point when a reflective wave from a peripheral blood vessel after a progressive pulse is generated in the aorta is added to the pulse waveform of the progressive wave and a highest systolic blood pressure value is represented by a ratio. As the rigidity of the artery increases, the reflective wave is quickly returned and thus the addition point is varied. Murgo [P. Murgo, N. Westerhof, J P. Giolma, S A. Altobelli] is a first person which researched the augmentation index for the first time. Murgo classified the pulse waveform into A, B, and C forms depending on a value of the augmentation index. Parameters affecting on the augmentation index are age, sex, heartbeat rate, blood pressure, and the like. Generally, as the age increases, the elasticity of the blood vessel is reduced and thus the adding time of the reflective wave to the systolic pulse is short, thereby increasing the augmentation index. The augmentation index of the aorta blood vessel can be accurately attained when measuring the pulse of the aorta blood vessel. However, this invasive method cannot be substantially applied to the actual clinical case and is very costly. To solve this limitation, researches for assuming an aortic waveform through a transfer function using the noninvasively measured in the radial artery and calculating the augmentation index from the assumed aortic waveform has been progressed [W W. Nichols, C H. Chen, B. Fetics].

As described above, the blood pressure value and the elasticity value of the artery are most importance indicators for the early diagnosis of the blood vessel-related diseases. Therefore, there is a need for a new measuring method for solving the limitations of the typical measuring methods. That is, in order to conveniently continuously measure the blood pressure of the aorta and the elasticity of the aorta without using the cuff but using the pulse of the radial artery of the wrist, a proper sensor should be first selected and a proper sensing technology should be applied.

### SUMMARY OF THE INVENTION

The present invention provides a sensing apparatus and method that can compensate for a variety of user conditions and a variety of use environmental conditions.

The present invention also provides a sensing apparatus and method that can remarkably improve the accuracy of a blood pressure measuring apparatus without using a cuff.

Embodiments of the present invention provide radial arterial pulse sensing apparatuses for noninvasive and continuous measurement of blood pressure and arterial elasticity, including: two pressure sensor for detecting radial arterial pulses: two cuffs that are disposed under the respective pressure sensors and expandable by application of external pressure; a motor unit for providing proper pressure to expandable pouches under the respective pressure sensors in a state where a wrist band having the pressure sensors is put on a wrist; a pulse wave velocity calculating unit that calculates a pulse wave transfer velocity to attain a blood pressure value using an output from the pressure sensors; and an augmentation index calculating unit that estimates the blood pressure value by finding a time point of a reflective wave of the pulses using the outputs from the pressure sensors.

In some embodiments, each of the pressure sensors may include a container and at least one sensing element in the container, wherein the containers of the pressure sensors are separated from each other to prevent the output of one of the pressure sensors from being affected by the pulses of a portion on which the other of the pressure sensors is located.

In other embodiments, each of the pressure sensors may include a container, at least one sensing element in the container, and an elastic member that is disposed on a contacting portion with the wrist to transfer the pulse, wherein each of the container is filled with a gel along which the pulses are transferred from the elastic member to the sensing elements.

In still other embodiments, the cuff may be located under the pressure sensor located on a radial artery and pressurized by the motor to press the radial artery.

In even other embodiments, the cuff may be pressurized step by step by 3-5 mmHg up to 30-50 mmHg to determine an optimal pressure and the pulses are detected while maintaining the determined optimal pressure.

In yet other embodiment, the optimal pressure applied to the wrist by the pressure sensor may be a pressure value to which a proper ratio of a mean pressure value of a pressure at a time point when a difference between the pulses is maximum and a pressure at a time point when a difference between the pulses is minimum as the pressure increases step by step is applied.

In further embodiments, the pulse wave velocity calculating unit may detect a starting point of the pulses at two portions under the optical pressure condition and calculates the pulse wave velocity using a distance between the sensors, thereby assuming an absolute value of a brachial blood pressure.

In other embodiments of the present invention, radial arterial pulse sensing methods for noninvasive and continuous measurement of blood pressure and arterial elasticity includes detecting an augmentation index representing a time point of a reflective wave from pulses detected under an optimal pressure condition; and providing a brachial blood pressure value from the augmentation index.

### BRIEF DESCRIPTION OF THE DRAWINGS

The accompanying drawings are included to provide a further understanding of the present invention, and are incorporated in and constitute a part of this specification. The drawings illustrate exemplary embodiments of the present invention and, together with the description, serve to explain principles of the present invention. In the drawings:

FIGS. 1A, 1B, and 1C are perspective views of a radial arterial pulse sensing apparatus for invasive and continuous measurement of blood pressure and arterial elasticity according to an embodiment of the present invention;

FIG. 2 show front and rear views of the radial arterial pulse sensing apparatus of FIGS. 1A to 1C;

FIG. 3 is a side view of the radial arterial pulse sensing apparatus of FIGS. 1A to 1C;

FIGS. 4A to 4C are views illustrating operation of the radial arterial pulse sensing apparatus of FIGS. 1A to 1C;

FIG. 5 is a block diagram of a system using the radial arterial pulse sensing apparatus of FIGS. 1A to 1C;

FIG. 6 is a flowchart illustrating operation of cuffs of the radial arterial pulse sensing apparatus of FIGS. 1A to 1C;

FIG. 7 is a graph illustrating calculation of an augmentation index from pulses detected by the radial arterial pulse sensing apparatus of FIGS. 1A to 1C;

FIG. 8 is a graph illustrating calculation of pulse wave velocity using pulses detected by the radial arterial pulse sensing apparatus of FIGS. 1A to 1C;

FIG. 9 is graphs illustrating a radial arterial pulse attained using the radial arterial pulse sensing apparatus of FIGS. 1A to 1C, an actual aortic pulse, and a aortic pulse assumed through a transfer function

FIG. 10 is a graph illustrating calculation of an augmentation index from a aortic pulse assumed by pulses detected by the radial arterial pulse sensing apparatus of FIGS. 1A to 1C.

### DETAILED DESCRIPTION OF PREFERRED EMBODIMENTS

Preferred embodiments of the present invention will be described below in more detail with reference to the accompanying drawings. The present invention may, however, be embodied in different forms and should not be constructed as limited to the embodiments set forth herein. Rather, these embodiments are provided so that this disclosure will be thorough and complete, and will fully convey the scope of the present invention to those skilled in the art.

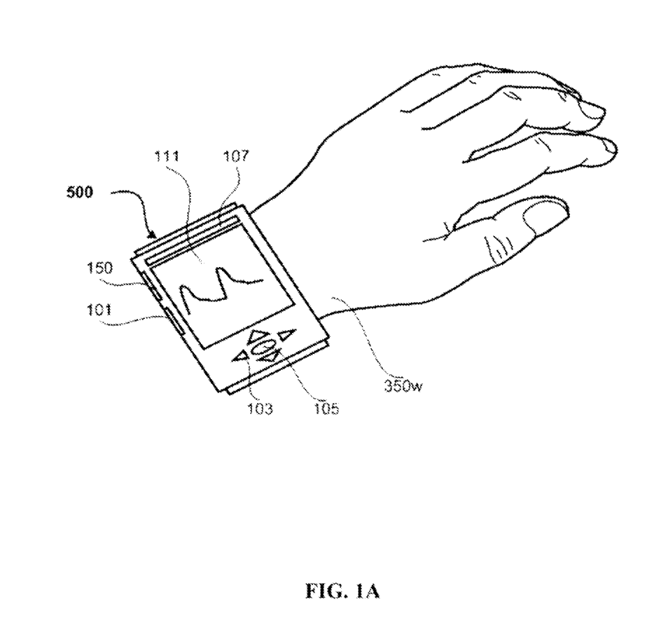

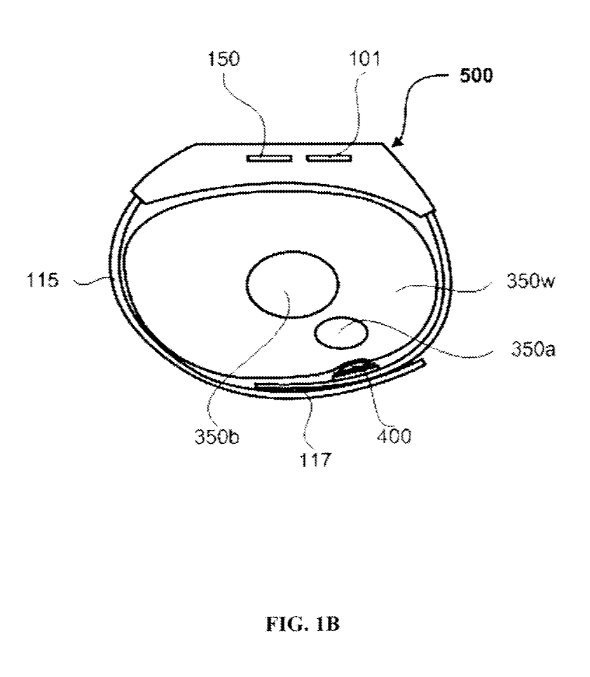

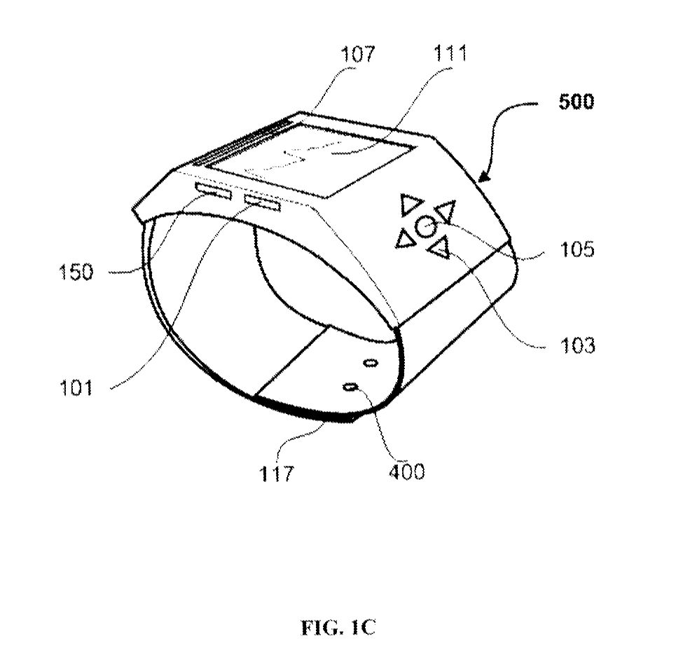

FIGS. 1A, 1B, and 1C are perspective views of a radial arterial pulse sensing apparatus for invasive and continuous measurement of blood pressure and arterial elasticity.

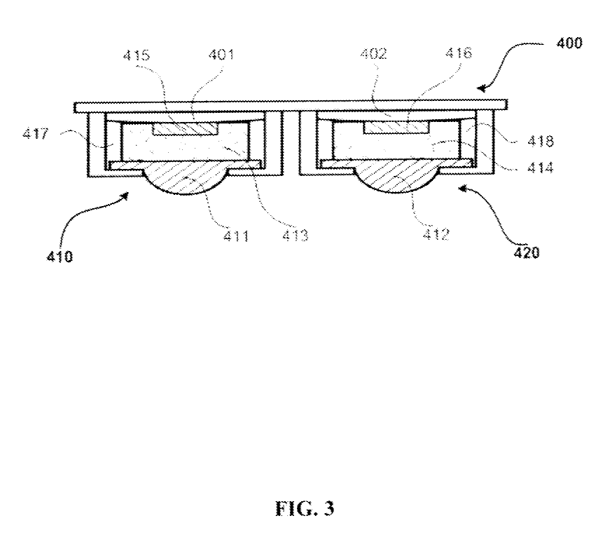

A noninvasive continuous blood pressure and arterial elasticity measuring apparatus 500 is put on a user's wrist 350w and operated by manipulating a power button 101, a menu button 103, and a selection button 105. An operational state is displayed on a light emitting diode 107. A signal detected by the apparatus 500 is displayed in real time on a display unit 111 as a waveform of a radial arterial pulse. When a wrist band 115 is wound around the wrist, a sensor unit 400 is located on the radial artery and applied force is automatically adjusted to an optimum state by cuffs 401 and 402 (see FIG. 3). The continuous pressure and arterial elasticity are calculated from the pulse detected by the apparatus and the calculated results may be transferred to an external device through a communication terminal 150.

The following will describe a method for measuring the continuous blood pressure and the arterial elasticity using the noninvasive continuous blood pressure and arterial elasticity measuring apparatus 500.

First, the sensor unit 400 is located on a radial artery portion 350a near a radial bone 350b of the wrist f the user.

Next, the wrist hand 115 is fastened by a Velcro-fastener 117 to have a proper pressure.

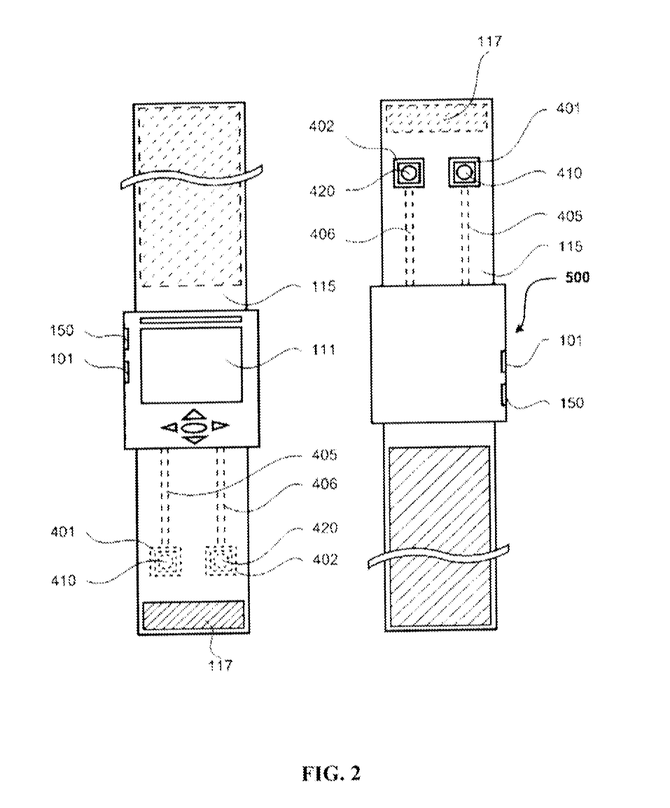

After the above, the blood measurement starts using the menu button 103 and the selection button 105. Pressure is applied to the cuffs 401 and 402 (see FIG. 2) applied to respective two sensors and pressure by which an optimum pulse can be detected is automatically set.

Next, when an optimum pressure value is set, the LED 107 operates and the pulse is detected.

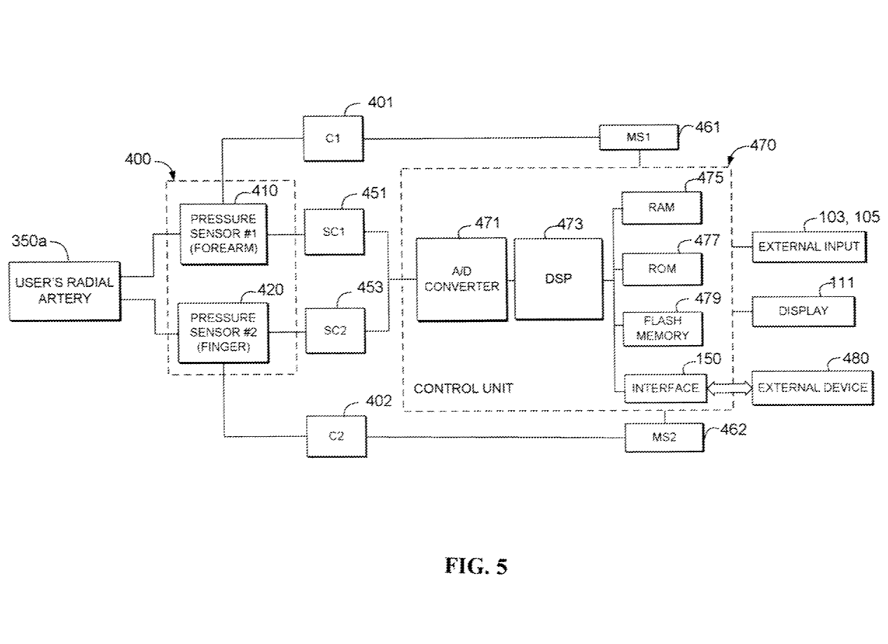

The detected pulse signal is operated through a series of processes in a control unit 470 (see FIG. 5) and a quantitative value such as the continuous blood pressure value and the arterial elasticity value are displayed on the display unit 111.

FIG. 2 is a view of the radial arterial pulse sensing apparatus of FIGS. 1A to 1C, illustrating front and rear portions when it is unfolded.

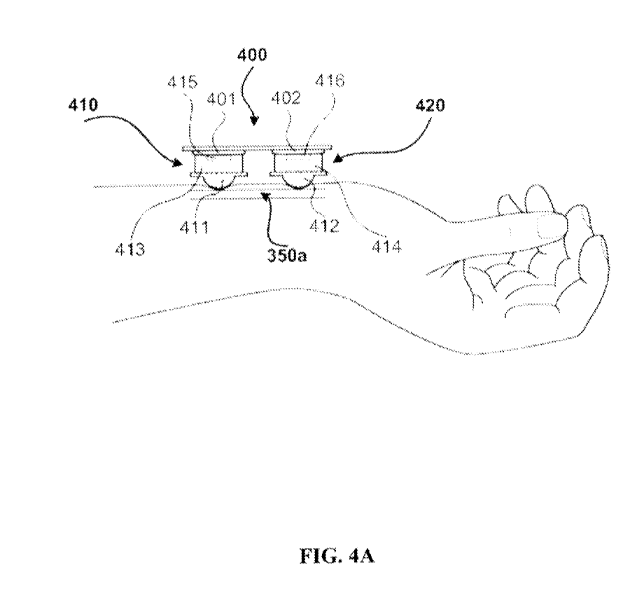

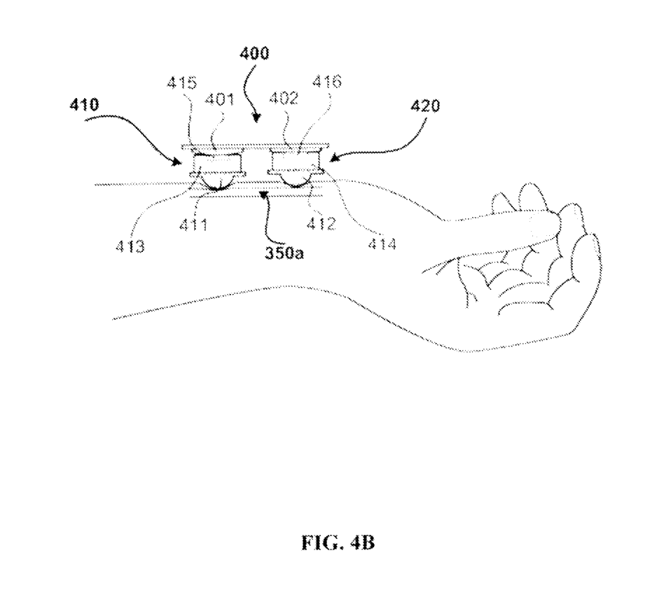

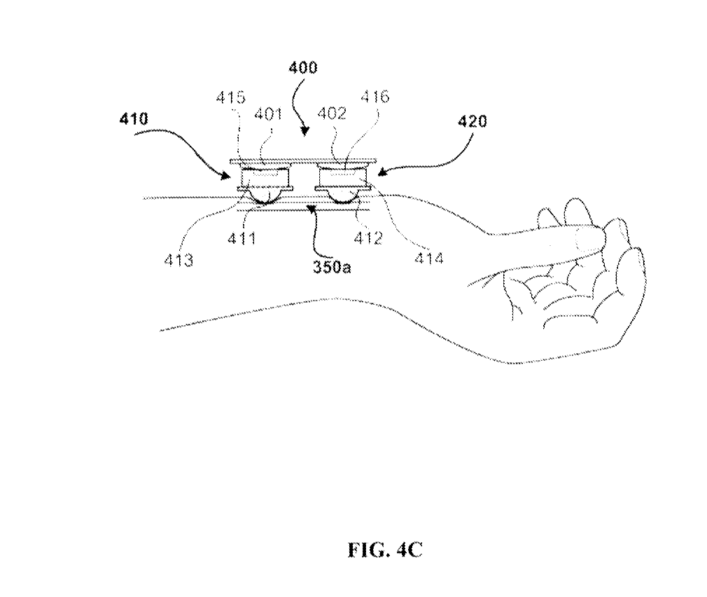

The noninvasive continuous blood pressure and arterial elasticity measuring apparatus 500 is put on the user's wrist 350w by the wrist band 115 such that the pressure sensors 401 and 402 can be located on the radial arterial portion 350a (see FIG. 4).

The cuffs 401 and 402 are disposed on respective upper ends of the pressure sensors 410 and 420. The pressure is applied to the cuffs 401 and 402 through air tubes 405 and 406 by a pressurizing motor disposed in the apparatus. The force for pressurizing the pressure sensor on the wrist is automatically adjusted to the optimal state by the cuffs 401 and 402.

FIG. 3 is a side view of the radial arterial pulse sensing apparatus of FIGS. 1A to 1C.

The sensor unit 400 has the pressure sensors 410 and 420. Sensing elements 415 and 416 of each pressure sensor are separated from each other so that the pressure applied by the artery pulse does not affect on the adjacent sensing element.

The pressure sensors 410 and 420 detect variation of force applied to the blood vessel wall as the flood flows along the blood vessel.

Elastic members 411 and 412 are disposed at portions where the sensor unit 400 contacts the radial artery 350a (see FIG. 4) to transfer generated pulse. Gels 413 and 414 are filled in sensor containers 417 and 418 to cover the sensing elements 415 and 416 so that the pulse signals can be transferred from the elastic members 411 and 412 to the sensing elements 415 and 416 along the gels 413 and 414.

The optimum pressure pressing the radial artery is set by analyzing relationship between pulse waveforms appearing by the operation of the cuffs 401 and 402 located on the respective pressure sensors by the motor in the apparatus 500. That is, the pulse signals detected by the sensing elements 415 and 416 of the pressure sensors 410 and 420 is utilized in correction of pressure that varies depending on wearing pressure of respective users.

FIGS. 4A to 4C are views illustrating operation of the radial arterial pulse sensing apparatus of FIGS. 1A to 1C.

The pressure sensors 410 and 420 detect variation of force applied to the blood vessel wall as the flood flows along the blood vessel.

FIG. 4A illustrates a state of the sensor unit 400 when the apparatus 500 is put on the wrist. When the apparatus 500 is initially put on the wrist, the pressure sensors 410 and 420 press the radial artery using same pressure as shown in FIG. 4A. When the apparatus 500 starts operating, as shown in FIG. 4B, only the cuff 401 of the sensor 410 located on a forearm operates to apply pressure of 5-10 mmHg to the sensor 410. After this, the pressure is increased step by step by 3-5 mmHg up to 30-50 mmHg. The pulse waves arc detected at each step and compared with each other.

The output values of the pressure sensors 410 and 420 are compared through the above-described process to set up the optimum pressure value pressing the radial artery 350a of the user. Next, as shown in FIG. 4C, the pulse waves are detected using the application pressures of the sensors 410 and 420. The pulse signals output from the pressure sensors 410 and 420 are operated by an operation program of the control unit 470 (see FIG. 5) and the operated signals are used to analyze the continuous blood pressure and the arterial elasticity.

FIG. 5 is a block diagram of a system using the radial arterial pulse sensing apparatus of FIGS. 1A to 1C.

The pressure sensors 410 and 420 are used to detect the pulse from the radial artery 350a of the user. When the apparatus 500 starts operating, the motor (MS1) 461 operates to apply pressure to the cuff (C1) 401. When predetermined pressure is applied, the outputs of the sensors 410 and 420 are converted into digital signals in the control unit 470 and analyzed. Next, the MS1 470 operates at each step and the pressure is applied to the C1 401. The outputs from the sensors 410 and 420 are converted into digital signals in the control unit 470 and analyzed. When the pressure application process in all of the predetermined steps is finished, the control unit 470 sets up an optimal pressure condition and the MS1 and MS2 461 and 462 operate to apply pressure values of the pressure condition to the C1 and C2 401 and 402. As described above, the radial artery is pressed with the optimal pressure and the blood pressure value is calculated using the pulse signal detected in this state.

The pulse signals detected by the pressure sensors 410 and 420 are amplified through analog signal processing units (SC1 and SC2) 451 and 453 and filtered, and then input to an analog input terminal of an analog/digital converter 471, after which being displayed on the display unit 11 and stored in a random access memory (RAM) 475. The pulse signals stored in the RAM 475 is processed by the digital signal processing unit 473 by the continuous blood and artery elasticity analyzing program stormed in a read only memory (ROM) 477. The analyzed results are displayed on the display unit 111 and stored in a flash memory 479. The resulting values of the stored continuous blood and artery elasticity may be transferred to the external device 480 through an interface terminal 150 such as a communication terminal.

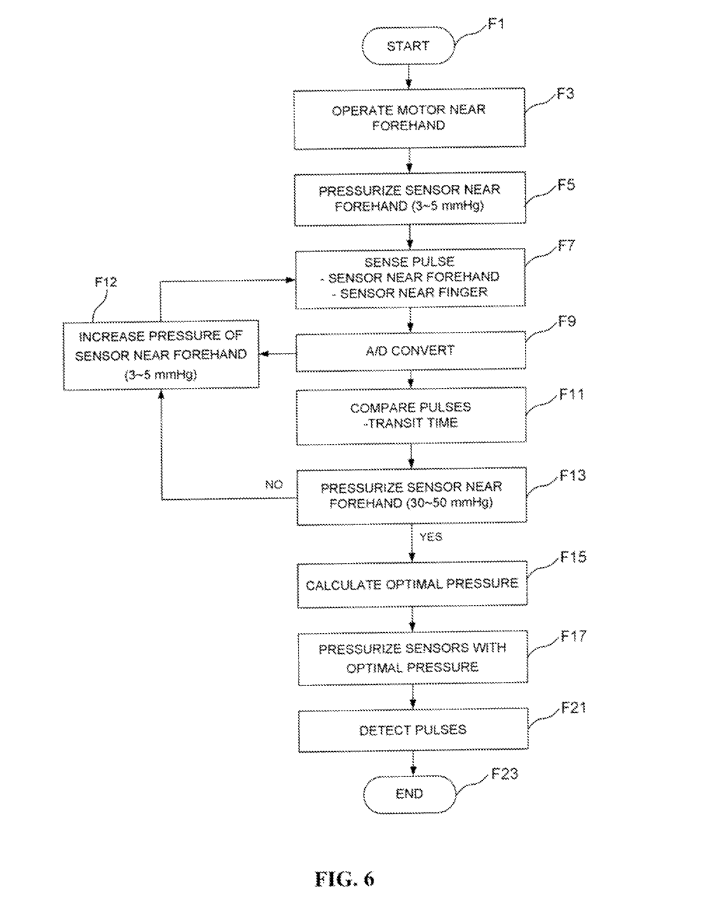

FIG. 6 is a block diagram illustrating the operation of the apparatus 500.

The pulse waves when the pressure application (F5) is realized by the operation (F3) of the motor connected to the sensor 410 near the forearm are detected and compared with each other. This uses a phenomenon where, as the pressure applied to the sensor 410 near the forearm increases, the pulse from the senor 420 near the finger is reduced. The pulse waves when the pressure sensor is pressurized at each step are detected (F7) and the detected signals are converted by the control unit 470 (see FIG. 5) (F9). The pulse waveforms are compared with each other by the internal algorithm (F11). The initial pressure is 3-5 mmHg and the pressure increases by 3-5 mmHg at each step (F12) up to 30-50 mmHg (F13). That is, the pressure starts from 5 mmHg and increases by 5 mmHg at each step up to 50 mmHg through 10 steps. The pulse waves are detected at each step. This process is for optimizing the radial artery and wrist conditions of each user. The optimum pressure condition (F15) is set as a pressure value that is attained by a proper ratio to a mean pressure value of a pressure value appearing at a time point where a difference between the pulse waves is maximized and a pressure value appearing at a time point where a difference between the pulse waves is minimized as the pressure on the forehand increases. When the optimum pressure value is set through the above-described process, the pressure sensor presses the radial artery with the set pressure (F17) and, at this point, the radial arterial pulse signals are detected (F21).

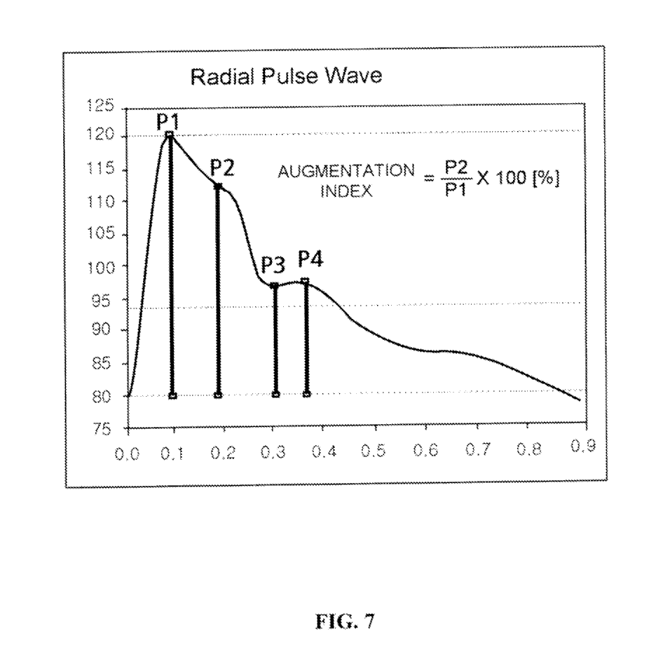

FIG. 7 is a graph illustrating calculation of an augmentation index from pulses detected by the radial arterial pulse sensing apparatus of FIGS. 1A to 1C;

The parameters detected include a highest diastolic point P1, an augmentation point P2, a notch point P3, and a highest systolic point P4.

Since a time point where the reflective wave is added to the radial arterial pulse occurs after the highest diastolic point P1, the notch point P3 for setting a section for detecting the augmentation point P2 is detected. In order to detect the notch point, a ranged from a maximum valley appearing after the highest diastolic point from a primarily differentiated mean pulse signal to a ⅔ point of the whole pulse is set. The number of zero-crossings having a positive gradient after the primarily differentiation is performed is identified. If the zero-crossing having the positive gradient exists, the zero-crossing point is defined as the notch point. If no zero-crossing having the positive gradient exists, a secondary differentiation is performed and the number of the zero-crossings having a negative gradient is identified.

If the zero-crossing having the negative gradient exists, the first zero-crossing point is defined as the notch point. If no zero-crossing having the negative gradient exists, a third differentiation is performed and the number of the zero-crossings having the positive gradient is identified. In addition, a zero-crossing point closest to the notch point detected from the radial artery pulse signal is deterred and this point is defined as the notch point.

A section from the highest diastolic point P1 to the notch point P3 is set to detect the augmentation point P2 and the number of the zero-crossing having the positive gradient after performing the primarily differentiation is identified. If the zero-crossing having the positive gradient exists, the zero-crossing point is defined as the augmentation point. If no zero-crossing having the positive gradient exists, a secondary differentiation is performed and the number of the zero-crossings having a negative gradient is identified.

If only one zero-crossing having the negative gradient exists, the zero-crossing point is defined as the augmentation point. If two or more zero-crossings having the negative gradient exist, the zero-crossing point having the highest valley among the valleys after the zero-crossing is defined as the augmentation point P2.

After the augmentation point is detected through the above-described process, a percentage of a ratio of the augmentation point to the highest diastolic point P1 is calculated and this percentage is set as a radial arterial pulse augmentation index.

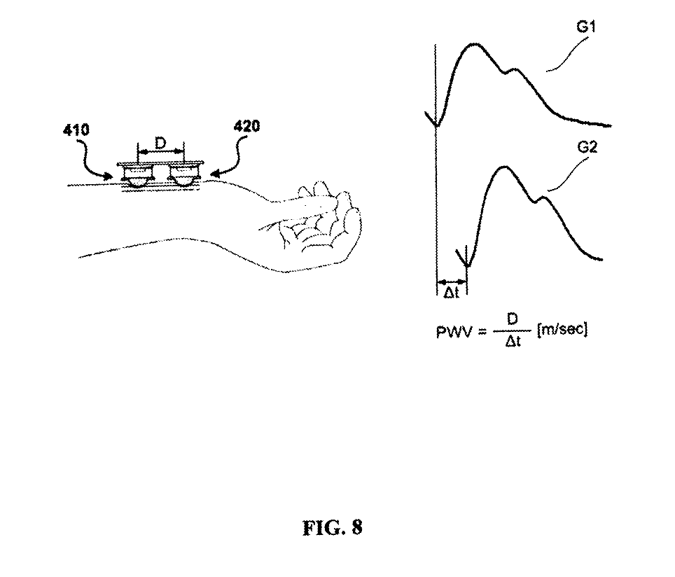

FIG. 8 is views illustrating calculation of pulse wave velocity using pulses detected by the radial arterial pulse sensing apparatus of FIGS. 1A to 1C;

The pulses are detected at two locations. Starting points of a waveform G1 detected from the sensor near the forearm and a waveform G2 detected from the sensor near the finger are detected and a time different Δt between the starting points is calculated. When dividing a distance D between the pressure sensors 410 and 420 by the time different Δt, the pulse wave velocity is attained.

When the elasticity of the blood vessel is reduced, the pulse wave velocity increases. Therefore, the pulse wave velocity may be an indicator reflecting the variation of the blood pressure and the blood vessel elasticity. Therefore, the correlation with the blood pressure value can be found using the pulse wave velocity.

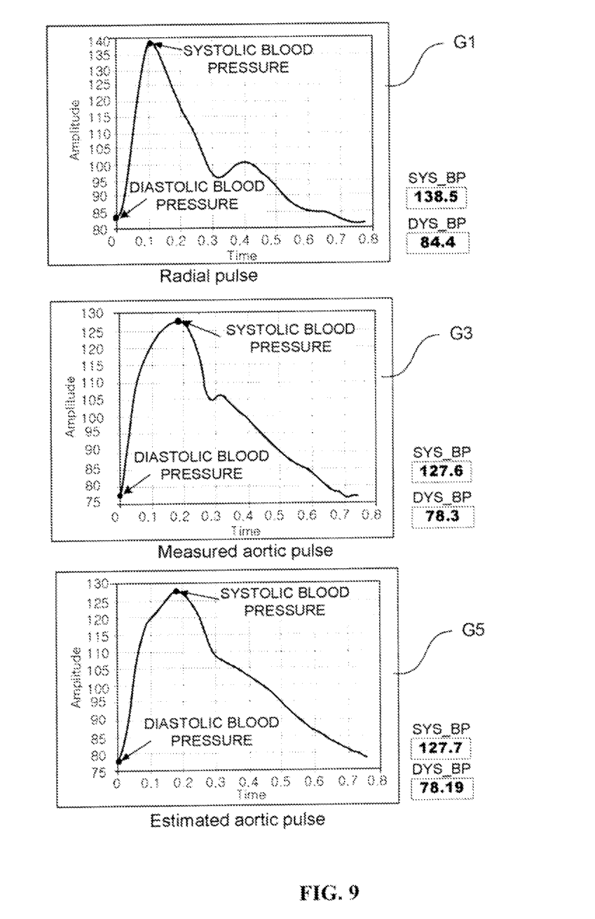

FIG. 9 is graphs illustrating a radial arterial waveform G1 detected by the radial arterial pulse sensing apparatus of the noninvasive continuous blood pressure and arterial elasticity measuring apparatus 500, a aortic pulse waveform P3 detected by the invasive method, and a aortic pulse waveform P5 assumed by applying a transfer function to the radial arterial pulse waveform.

In order to apply the detected radial arterial pulse wave to an ARX model, the radial arterial pulse waveform G is applied as an input and the aortic pulse waveform G2 is applied as an output and the coefficient of the ARX model is calculated using a least square method. In the ARX model, the transfer function coefficient using the least square method. The least square method is for calculating coefficients that is smallest when squaring a difference between an actual output and a calculated output. This can he expressed by the following equation 1.

$$\frac{1}{N} {\sum }_{t = 1}^{N} {( y ( t ) - {y}^{} ( t / θ ) )}^{2} = \frac{1}{N} {\sum }_{t = 1}^{N} {( y ( t ) - {φ}^{T} ( t ) θ )}^{2} \qquad [ Equation 1 ]$$

Equation 1 is differentiated with respect to θ to obtain a transfer function coefficient \hat{θ}_{N} which allows the differentiated equation 1 to be 0. The transfer function coefficient \hat{θ}_{N} can be expressed by the following equation 2.

$${{θ}^{}}_{N} = {[ {\sum }_{t = 1}^{N} φ ( t ) {φ}^{T} ( t ) ]}^{- 1} {\sum }_{t = 1}^{N} φ ( t ) y ( t ) \qquad [ Equation 2 ]$$

Since the radial arterial pulse is detected and the highest and lowest blood pressure values of the brachial artery are calculated from the radial arterial pulse, the highest and lowest blood pressure values of the aortic pulse that is assumed using the transfer function can be calculated by applying an equivalent ratio.

Generally, for healthy persons, the blood pressure value is gradually lowered as the measuring portion is increasing spaced apart from the heart. For elderly persons, a difference between the aortic blood pressure value and the peripheral blood pressure value is greater. Therefore, the blood vessel age can be assumed using this.

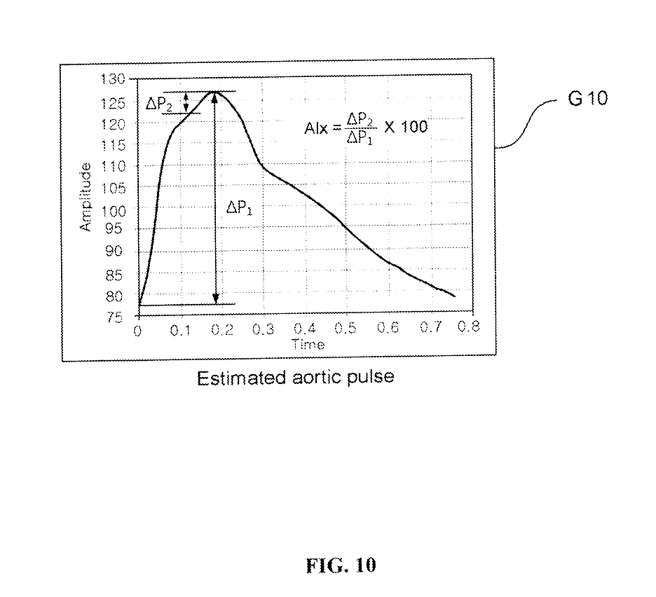

FIG. 10 illustrates a calculation of the augmentation index for providing the aortic elasticity from the aortic pulse G10 that is assumed using the radial arterial pulse waveform detected by the radial arterial pulse sensing apparatus used in the noninvasive continuous blood pressure and arterial elasticity measuring apparatus 500 of the embodiment.

After the aortic pulse waveform is primarily differentiated, the number of the zero-crossings having the positive gradient is identified. If only one zero-crossing having the positive gradient exists, this zero-crossing point is defined as the augmentation point. If two or more zero-crossings having the positive gradient exist, the zero-crossing point after the highest valley among the valleys appearing before the zero-crossing is defined as the augmentation point. If no zero-crossing exists, a secondary differentiation is performed to identify the number of the zero-crossings having the positive gradient.

If only one zero-crossing having the positive gradient exists, this zero-crossing point is defined as the augmentation point. If two or more zero-crossings having the positive gradient exist, the highest peak and the second highest peak among the peaks appearing after the zero-crossing are compared with each other. When the second highest peak is greater than 20% of the highest peak, the highest peak point is defined as the augmentation point. When the second highest peak is less than 20% of the highest peak, a cut-off frequency of a digital low band pass filter is reduced from 20 Hz to 15 Hz and applied to the mean pulse signal and the secondary differentiation is performed again. If no zero-crossing having the positive gradient exists, a third differentiation is performed to identify the number of the zero-crossings having the negative gradient.

If only one zero-crossing having the negative gradient exists, this zero-crossing point is defined as the augmentation point. If two or more zero-crossings having the negative gradient exist, the zero-crossing point after the highest valley among the valleys appearing after the zero-crossing is defined as the augmentation point.

If no zero-crossing exists, it is regarded that no augmentation point exists before the systolic peak point. In addition, the augmentation index after the systolic peak point is detected. After the augmentation point is found through the above-described process, a ratio of a different between the systolic peak value and the augmentation point peak value to the systolic peak value is calculated and the percentage of this is defined as the augmentation index.

According to the embodiment, a sensing technology that can dramatically improve inaccuracy of the blood pressure measuring apparatus is applied. In addition, the sensing apparatus sand method of the embodiment can be utilized to develop a system for early diagnosis of the specific disease related to a cardiovascular system such as the brachial artery, the central aorta, and the blood vessel elasticity by providing pulse signals for providing the accurate blood pressure value and arterial elasticity considering a variety of user conditions and a variety of use conditions.

What is claimed is:

1. A radial arterial pulse sensing apparatus for noninvasive and continuous measurement of blood pressure and arterial elasticity, comprising:
two pressure sensor for detecting radial arterial pulses;
two cuffs that are disposed under the respective pressure sensors and expandable by application of external pressure;
a motor unit for providing proper pressure to expandable pouches under the respective pressure sensors in a state where a wrist band having the pressure sensors is put on a wrist;
a pulse wave velocity calculating unit that calculates a pulse wave transfer velocity to attain a blood pressure value using an output from the pressure sensors; and
an augmentation index calculating unit that estimates the blood pressure value by finding a time point of a reflective wave of the pulses using the outputs from the pressure sensors.

2. The radial arterial pulse sensing apparatus of claim 1, wherein each of the pressure sensors includes a container and at least one sensing element in the container, wherein the containers of the pressure sensors are separated from each other to prevent the output of one of the pressure sensors from being affected by the pulses of a portion on which the other of the pressure sensors is located.

3. The radial arterial pulse sensing apparatus of claim 1, wherein each of the pressure sensors includes a container, at least one sensing element in the container, and an elastic member that is disposed on a contacting portion with the wrist to transfer the pulse, wherein each of the container is filled with a gel along which the pulses are transferred from the elastic member to the sensing elements.

4. The radial arterial pulse sensing apparatus of claim 1, wherein the cuff is located under the pressure sensor located on a radial artery and pressurized by the motor to press the radial artery.

5. The radial arterial pulse sensing apparatus of claim 1, wherein the cuff is pressurized step by step by 3-5 mmHg up to 30-50 mmHg to determine an optimal pressure and the pulses are detected while maintaining the determined optimal pressure.

6. The radial arterial pulse sensing apparatus of claim 1, wherein the optimal pressure applied to the wrist by the pressure sensor is a pressure value to which a proper ratio of a mean pressure value of a pressure at a time point when a difference between the pulses is maximum and a pressure at a time point when a difference between the pulses is minimum as the pressure increases step by step is applied.

7. The radial arterial pulse sensing apparatus of claim 1, wherein the pulse wave velocity calculating unit detects a starting point of the pulses at two portions under the optical pressure condition and calculates the pulse wave velocity using a distance between the sensors, thereby assuming an absolute value of a brachial blood pressure.

8. A radial arterial pulse sensing method for noninvasive and continuous measurement of blood pressure and arterial elasticity, the method comprising:
detecting an augmentation index representing a time point of a reflective wave from pulses detected under an optimal pressure condition; and
providing a brachial blood pressure value from the augmentation index.
